(19) United States
(12) Patent Application Publication
(10) Pub. No.: US 2005/0208050 A1
(43) Pub. Date: Sep. 22, 2005

(54) Compounds for the diagnosis/prevention/treatment of alzheimer's disease

(76) Inventors: Gerd Multhaup, Kleinmachnow (DE); Stefan Scheuermann, Hirschberg (DE); Konrad Beyreuther, Heidelberg (DE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 10/494,934

(22) PCT Filed: Nov. 8, 2002
(86) PCT No.: PCT/EP02/12523

§ 371 Date: Apr. 29, 2005

Related U.S. Application Data
(60) Provisional application No. 60/331,175, filed on Nov. 9, 2001.

(51) Int. Cl. C12Q 1/68; G01N 33/53; G01N 33/567; C07H 21/04; A61K 39/395; C12N 9/64; C12N 15/09

(52) U.S. Cl. 424/145.1; 435/6; 435/7.2; 435/69.1; 435/226; 435/320.1; 435/325; 530/388.26; 536/23.2

(57) ABSTRACT

The present invention relates to the homodimerization of the amyloid precursor protein (APP) and the relevance of this process for the presence of enhanced amounts of the amyloid β-peptide (Aβ) in vivo. The present invention provides novel compounds useful for the diagnosis/prevention/treatment of diseases like Alzheimer's disease. Also provided are vectors, host cells and antibodies. The invention further relates to diagnostic and therapeutic methods useful for diagnosing and treating Alzheimer's disease.

### FIELD OF THE INVENTION

The present invention relates to homodimerization of the amyloid precursor protein (APP) and the relevance of this process for presence of enhanced amounts of amyloid β-peptide (Aβ) in vivo and particularly to novel compounds useful for the diagnosis/prevention/treatment of diseases like Alzheimer's disease.

### BACKGROUND OF THE TECHNOLOGY

Although much progress has been made over the past few years regarding the involvement of the amyloid precursor protein (APP) in Alzheimer's disease (Neve et al., Brain Res. 886 (2000), 54-66), the biological function of cellular APP still remains enigmatic. APP is part of a super-family from which sixteen homologous amyloid precursor-like proteins (APLP) and APP species homologues are derived. APP is an ubiquitous glycoprotein expressed highly in neurons. APP deficient mice are viable and develop normally, but they display minor defects that differ according to the null strain, e.g., copper levels are increased in the cerebral cortex and liver of APP and APLP2 knockout mice. Thus, APP is not required for the expression of a critical cell function, but may be involved in the modulation of neuronal functions at the cellular level. The structure of APP resembles a cell-surface receptor (Kang et al., Nature 325 (1987), 733-6). APP has a function in cell-cell and cell-matrix interactions that is evident by binding to heparin and collagen. Soluble forms of APP promote neurite outgrowth and cell adhesion and are neuroprotective. Surprisingly, the ectodomain of APP is phosphorylated within the acidic region in the N-terminal half of the molecule. The phosphorylation sites were mapped to serine residues 189 and 206 within the zinc(II)binding site of APP. The ectodomain phosphorylation of APP is inhibited by heparin, most likely by steric hindrance through binding of heparin to the N-terminal domain. Likewise, heparin also inhibits APP-APP interaction in vitro.

It has been shown that in brain a distinct percentage of APP is present on the cell surface as a membrane protein of type I. This cell surface APP may mediate the transduction of extracellular signals into the cell via its C-terminal tail. Involvement of APP in neuronal development, synaptogenesis, and synaptic plasticity indicated that the observed function is not restricted to secreted APP, raising the possibility that some aspects of synaptic plasticity are mediated by cell-associated APP.

APP is found on the cell surface in neurons possessing a neurite-outgrowth activity. It was shown that APP binds to the brain-specific signal transducing G protein G₀. Interestingly, APP mutants of familial Alzheimer's disease (FAD) can cause Gₒ-mediated apoptosis in neuronal cells and these same FAD mutants cause the intracellular accumulation of C100.

Dimerization of receptors induced by ligand binding is a common means of triggering signal transduction in mammalian cells. Dimerization appears to play a role in cytokine receptor signaling and conformational changes brought about by receptor oligomerization in response to ligand binding are likely to activate the tyrosine kinase receptors. Evidence that dimerization of proteins involved in neurodegenerative processes could play a role in the disease state is derived from current models of the role of PrP in scrapie pathogenesis. Oligomeric forms of PrP are believed to facilitate a more rapid conversion of PrP^{c} into PrP^{Sc} and were observed in scrapie-infected hamster brains.

Up to now, there has been neither a clear evidence that APP can homodimerize nor that homodimerization of APP is directly linked to amyloid Aβ production and therefore is a risk factor for the development of the disease. Due to the fairly unknown etiology of Alzheimer's disease there is currently no therapy available against the cause of the disease. This could be based on the inhibition of amyloid Aβ production that would eliminate the formation of senile plaques.

The above discussed limitations and failings of the prior art to provide meaningful compounds which are of therapeutic (or diagnostic) value, has created a need for markers which can be used diagnostically, prognostically and therapeutically over the course of diseases like AD.

The present invention fulfills such a need: In the experiments leading to the present invention it was investigated whether cellular APP exists as a homodimer and whether dimerization can promote or attenuate the Aβ production in the β-secretory pathway of APP. Based on surface cross-linking studies it was found that APP homodimerizes efficiently in the ER and Golgi and is secreted as a soluble protein from eukaryotic cells and from yeast cells as a recombinant protein. A mutationally induced dimerization of cellular APP produced a 7-fold increase of soluble Aβ which is central to the pathogenesis of the disease. This suggests that dimerization-mediated regulation of APP processing is likely to be physiologically relevant. APP may exist as a homodimer on the cell surface. The homodimer is stabilized as an inactive dimer by multiple dimerization interfaces. A dimerization in this manner would provide a potential mechanism for a negative regulation of proposed APP functions and a concomitant increase of amyloid formation. Accordingly, the inhibition of APP homodimerization should result in an decrease of amyloid formation and, thus, useful for the prevention and/or treatment of Alzheimer's disease.

### SUMMARY OF THE INVENTION

The present invention is based on the finding that cellular APP exists as homodimer matching best with a two site model. Consistent with published crystallographic data, it could be shown that a deletion of the entire sequence after the KPI domain did not abolish APP homodimerization suggesting that two domains are critically involved but that neither is essential for homodimerization. Finally, stabilized dimers were generated by expressing mutant APP with a single cysteine in the ectodomain juxtamembrane region. Mutation of Lys624 to cysteine produced approximately 6-8-fold more Aβ than cells expressing normal APP. These results suggest that amyloid Aβ production can in principle be positively regulated by dimerization in vivo. Accordingly, homodimerization might be a physiologically important mechanism for regulating the proposed signal activity of APP, i.e. the possible caustative mechanism involved in the development of Alzheimer's disease.

The present invention, thus, provides a peptide-homodimer (and corresponding DNA sequences) comprising amino acids 17 to 40 of the amyloid β-peptide and antibodies raised by using this peptide as an antigen, which can be used diagnosticall.

In one embodiment, the present invention provides compounds which inhibit APP homodimerization.

In another embodiment, the present invention provides a diagnostic method for detecting a disease associated with the presence of an enhanced amount of amyloid-β-peptide, e.g. Alzheimer's disease.

In another embodiment, the present invention provides a method for preventing, treating or ameliorating a disease associated with the presence of an enhanced amount of amyloid-β-peptide, e.g. Alzheimer's disease.

Finally, the present invention provides a method for identifying compounds which are useful for therapy, i.e. which are capable of inhibiting the homodimerization of APP.

### BRIEF DESCRIPTION OF THE DRAWINGS

FIG. 1: Disulfide linked oligomers of [³⁵S]-methionine or [³⁵S]-sulfate labeled APP₆₉₅ are found in cell membranes.

Samples of enriched APP were diluted and cross-linked (+) or not cross-linked (−) with 0.4 mM of the cleavable cross-linker DTSSP, as indicated (A). Metabolically labeled APP was purified by immunoprecipitation from solubilized SY5Y neuroblastoma cell membranes with anti-APP (22734). The migration positions of monomeric APP (−110 kDa), dimeric APP (−190 kDa) and tetrameric APP (−400 kDa) are indicated on the right with arrowheads. Samples were analyzed under nonreducing (A) and reducing conditions (B) on 7-10% acrylamide gradient gels. The bands from the unreduced samples were cut from the gel (A), eluted, reduced, and separated in a second SDS-PAGE to identify their individual constituents (B). APP migrates as a monomer (closed arrowheads) under reducing conditions, independent from the initial oligomeric form, if either derived from a dimer (grey arrowheads) or a tetramer (open arrowheads). Note that a fraction of the protein migrating at the position of dimers and tetramers is still present on the SDS gel after treatment with reducing agents. The fluorographed gels were exposed at −80° C. for at least 6 days (B). The positions of the molecular weight standards are given on the left.

FIG. 2: Recombinant APP₁₈₋₃₅₀ elutes from a gel filtration column preferentially as a homodimer

Fractions of yeast APP₁₈₋₃₅₀ eluting from a Q-Sepharose column were subjected to gel filtration by FPLC using a Superdex 200 column, with online recording of absorbance at 280 nm. 2-ml fractions were collected and analyzed for their protein concentration (A) and by SDS-PAGE followed by Coomassie Blue staining (B). Apparent molecular weights of APP₁₈₋₃₅₀ eluting form the gel permeation column were calculated from the logarithmic curve of molecular weights (MW) of the standards spread against the fraction number (C). Note the correspondence between Coomassie Blue-stained APP₁₈₋₃₅₀ (peak in fractions 33-37) and the protein concentration shown in (A). SDS-PAGE showed that the major peaks contained APP₁₈₋₃₅₀ with an apparent molecular mass of 50 kDa whereas the majority of APP eluted from the gel permeation column at a size of 113 kDa (C).

FIG. 3: Immunoblotting analysis of cross-linked APP₁₈₋₃₅₀ with polyclonal anti-APP₈₋₃₅₀

The homobifunctional cross-linkers BS³ (A) and DTSSP (B) were used on 10 μg APP₁₈₋₃₅₀ from pooled gel permeation fractions (33-37, shown in FIG. 2B) in concentrations as indicated. BS³/DTSSP: C, mock cross-linking without BS³ and DTSSP. The positions of monomer (closed arrowhead), dimer (grey arrowhead) and tetramer (open arrowhead) are indicated at the right side of the Figure. The positions of molecular-weight markers (in kilodaltons) are indicated on the left side of the Figure. Similar labels are used throughout the study.

FIG. 4. Oligomeric states of APP₆₉₅-K624C mutant APP and Aβ production thereof.

Dimerization of APP695-K624C mutant APP is mediated by the presence of the disulfide bond in the juxtamembrane domain of APP. Western blotting with monoclonal antibody 22C11 of the immunoprecipitated Cys-mutant APP (APP₆₉₅-K624C) but not the wild-type APP from lysates of stably transfected SY5Y cells reveals dimers (˜220 kDa; myc-/myc-APP, open arrow) under non-reducing conditions (-DTT), separated on 7-10% acrylamide gradient gels (A). In the presence of DTT, APP dimers are converted to monomers (lane APP₆₉₅-K624C, +DTT; myc-APP, open arrow head) migrating at about 110 Kda and co-migrating with endogenous forms of APP from SY5Y cells (A). Amyloid Aβ was immunoprecipitated with monocional antibody WO-2 from the medium of SY5Y cells transfected as described in A (B), separated on NuPAGE-gels under non-reducing (lanes -DTT/APP₆₉₅-K624C, -DTT/APP₆₉₅-Wt) or reducing conditions (+DTT/APP₆₉₅-K624C, +DTT/APP₆₉₅-wt) and analyzed by Western blotting with the same antibody WO-2 after cutting the filter (B). Under non-reducing conditions (-DTT), Aβ dimers (−8.5 kDa) can be precipitated from cell culture supernatant of transfected APP₆₉₅-K624C SY5Y cells (B). In the presence of DTT, Aβ dimers are converted to monomers (lane APP₆₉₅-K624C, +DTT) migrating at −4.5 kDa (B). Quantification of Aβ production was based on the average of three replicates. Note the faint bands of Aβ derived from endogenously expressed APP (B).

Oligomerization of N-terminally extended APP forms was assessed in lysates from transiently transfected COS-7 cells after immunoprecipitation with polyclonal anti-APP₁₈₋₃₅₀ (40090) and Western blotting with 22C11 under non-reducing conditions (C). From top to bottom, the positions of myc-K624C/GFP-K624C APP₆₉₅ heterodimers from doubly transfected cells (closed arrow), myc-K624C APP₆₉₅ homodimers (open arrow) in myc-K624C/GFP-K624C doubly and mycK624C transfected cells, endogenous KPI forms of APP from COS-7 cells (star) in all lines, monomeric GFP-K624C (closed arrow head) in myc-K624C/GFP-K624C and GFP-K624C transfected cells and monomeric myc-K624C (open arrow head) in myc-K624C/GFP-K624C and myc-K624C transfected cells are shown. Note that homodimers of GFP-K624C were not detected.

FIG. 5: Schematic representation of the K624C mutant of APP₆₉₅

The transmembrane domain is indicated in yellow and Aβ in red. The part of Aβ that is inserted in the membrane is enlarged. Amino acid residues of Aβ are given in the one letter code. Note that the K624C mutation is located at the juxtamembrane position.

FIG. 6: Dimerization of APP₁₈₋₃₅₀

FIG. 7: Immunostaining with antibody 40090
A. Immunostaining of APP695 transfected COS-7 cells with polyclonal serum 40090 which has been produced in rabbits against APP18-350
B. Lightmicroscopic presentation of the cells shown in A oberlapped with the immunostaining
C. Immunostaining of APP695_{K624C} transfected COS-7 cells with polyclonal serum 40090 which had been produced in rabbits against APP18-350
D. Lightmicroscopic presentation of the cells shown in C oberlapped with the immunostaining

Method used for staining described in Kins-Setal, Nat. Neurosci. 2000, 3, pp. 22-29.

FIG. 8: Dimerization of APP-K624C

FIG. 9: Immunostaining of APP and APP K624C transfected COS cells
left panel: immunostaining of APP695 transfected cells
a. APP695 in enodplasmatic reticulum (ER), in vesicular structures and cytoplasma, stained with polyclonal antibody AK40090 which had been generated against APP18-350
b. APP695 which had been stained with monoclonal anti-myc antibody appears in vesicular structures und in a substantially cytoplasmic distribution of the APP
c. APP695 which has been stained with polyclonal antibodies MX-02/MX-03 is substantially located in the plasma membrane. The staining of the membrane located APP by the serum MX-02/MX-03 is caused by the immunosation with the dimer p3₁₇₋₄₀.

right panel: immunostaining of APP695_{K624C} transfected cells
d. Konstitutively formed APP dimers which are preferably formed by APP695_{K624C} are recognized in the plasma membrane by pAβ40090 since this antibody is directed against APP18-350 dimers. In view of the used construct APP695_{K624C} much more APP dimers are present in part d. than in part a. pAβ40090 shows here a preferred staining of membrane located APP695_{K624C}.
e. Cells stained with monoclonal myc antibody show no significant difference to the staining in part b. since this antibody recognizes an epitope at the N-terminal of APP but is unable to distinguish between APP monomers and dimers.
f. The polyclonal antibody MX-O₂/MX-03 shows substantially a staining of APP dimers in the plasma membrane of APP⁶⁹⁵K624C transfected COS-7 cells which are quite similar to the APP695 transfected cells in part c.

FIG. 10: Dimerization of APP: first step to Aβ

FIG. 11: APP dimerisation—inhibition through synthetic peptide (APP amino acids 96-116)

Recombinant APP (expressed in Pichia pastoris) elutes as SDS-stable homogenous dimer from a gel permeation column (Scheuermann et al., J. of Biol. Chem., Vol. 276, No. 36, pp. 33923-33929). In order to test whether the loop region of APP (AS 96-114) can inhibit dimerisation, crosslinking experiments have been performed using dimer containing fractions from a gel permeation column. The peptide was synthesized and added in the indicated molar fractions (8, 16, 82) to APP18-350 before DTSSP as crosslinker was added. Controls are C1: without addition of synthetic peptide; C2: without crosslinking agent, the addition of peptide without intramolecular disulfide bridge (both Cys are mutated to Ser) and the homologue loop-peptide from APLP2 (AS 114 to 132) with an intact disulfide bridge. The result is that only homologous peptides from APP and APLP2 with intact disulfide bridges can inhibit the dimerisation/tetramerisation of APP18-350 (monomer at 40 kDa).

The abbreviations used are: APP, amyloid precursor protein; APLP, amyloid precursor-like protein; BS, bis(sulfosuccinimidyl)propionate; CBP, collagen binding peptide; DTSSP, dithiobis(sulfosuccinimidyl)propionate; FAD, familial Alzheimer's disease; Aβ, amyloid β-peptide; ER, endoplasmic reticulum; KPI, kunitz protease inhibitor; DTT, dithiothreitol; PrP, prion protein; PTPs, protein tyrosine phosphatases.

### DETAILED DESCRIPTION OF THE INVENTION

Before the present invention is described in further detail, it is understood that this invention is not limited to the particular methodology, protocols, cells lines, vectors and reagents described as these may vary. It is also to be understood that the terminology used herein is for the purpose of decribing particular embodiments only, and is not intended to limit the scope of the present invention which will be limited only by the appended claims.

It must be noted that as used herein and in the appended claims, the singular forms of “a”, “an” and “the” include plural reference unless the context dictates otherwise.

Thus, for example, reference to “a host cell” includes a plurality of host cells, reference to the “antibody” is a reference to one or more antibodies and equivalents thereof known to those skilled in the art, and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the methodologies which are reported in the publications which might be used in connection with the invention. Nothing herein is to be construed as an admission that the invention is not entitled to antidate such an disclosure by virtue of prior invention.

The present invention relates to an isolated nucleic acid molecule encoding a peptide comprising amino acids 17 to 40 of the amyloid β-peptide, wherein the peptide is selected from the group consisting of
(a) a peptide wherein at least one of the naturally occuring amino acids is replaced by a cysteine residue;
(b) a peptide wherein at least one cysteine residue is inserted into the naturally occuring amino acid sequence; and
(c) a peptide which is a fragment, derivative or allelic variation of a peptide of (a) or (b).

As used herein, the term “isolated nucleic acid molecule” includes nucleic acid molecules substantially free of other nucleic acids, proteins, lipids, carbohydrates or other materials with which it is naturally associated.

In a first embodiment, the invention provides an isolated nucleic acid molecule encoding a peptide comprising amino acids 17 to 40 of the amyloid β-peptide (LVFFAEDVGSNKGAIIGIMVGGVV) wherein at least one of the naturally occuring amino acids is replaced by a cysteine residue.

In another embodiment, the invention provides an isolated nucleic acid molecule encoding a peptide consisting of amino acids 17 to 40 of the amyloid β-peptide (LVFFAEDVGSNKGAI-IGLMVGGVV) wherein at least one cysteine residue is inserted into the naturally occuring amino acid sequence.

The nucleic acid molecules of the invention can be both DNA and RNA molecules. The nucleic acid molecules of the invention can be isolated from natural sources or can be synthesized according to known methods.

In a further embodiment, the present invention provides nucleic acid molecules encoding a fragment, derivative or allelic variation of the above peptide which have substantially the same immunogenic properties, i.e. can be used for generating an antibody specifically binding to an APP- or Aβ-homodimer. “Fragments” are understood to be parts of the nucleic acid molecules that are long enough to encode a peptide which can be used as an immunogene. The term “derivative” in this context means that the sequences of these molecules differ from the sequences of the nucleic acid molecules described above at one or several positions but have a high level of homology to these sequences. Homology hereby means a sequence identity of at least 60%, in particular an identity of at least 80%, preferably of more than 90% and particularly preferred of more than 95%. These peptides encoded by the nucleic acid molecules have a sequence identity to the amino acid sequence LVFFAEDVGSNKGAIIGLMVGGVV of at least 80%, preferably of 85% and particularly preferred of more than 90%, 95%, 97% and 99%. The deviations to the above-described nucleic acid molecules may have been produced by deletion, substitution, insertion or recombination.

The nucleic acid molecules that are homologous to the above-described molecules and that represent derivatives of these molecules usually are variations of these molecules that represent modifications having substantially the same immunogenic function. They can be naturally occurring variations, for example sequences from other organisms, or mutations that can either occur naturally or that have been introduced by specific mutagenesis. Furthermore, the variations can be synthetically produced sequences. The allelic variants can be either naturally occurring variants or synthetically produced variants or variants produced by recombinant DNA processes.

Generally, by means of conventional molecular biological processes it is possible (see, e.g., Sambrook et al., 1989, Molecular Cloning, A Laboratory Manual, 2^{nd} edition Cold Spring Harbor Laboratory Press, Cold Spring Harbor, N.Y.) to introduce different mutations into the nucleic acid molecules of the invention.

For the manipulation in prokaryotic cells by means of genetic engineering the nucleic acid molecules of the invention or parts of these molecules can be introduced into plasmids allowing a mutagenesis or a modification of a sequence by recombination of DNA sequences. By means of conventional methods (cf. Sambrook et al., 1989, Molecular Cloning: A Laboratory Manual, 2^{nd} edition, Cold Spring Harbor Laboratory Press, NY, USA) bases can be exchanged and natural or synthetic sequences can be added. In order to link the DNA fragments with each other adapters or linkers can be added to the fragments. Furthermore, manipulations can be performed that provide suitable cleavage sites or that remove superfluous DNA or cleavage sites. If insertions, deletions or substitutions are possible, in vitro mutagenesis, primer repair, restriction or ligation can be performed. As analysis method usually sequence analysis, restriction analysis and other biochemical or molecular biological methods are used.

The peptides encoded by the various variants etc. of the nucleic acid molecules of the invention show certain common characteristics, such as immunological reactivity.

In a preferred embodiment, the present invention relates to an isolated nucleic acid molecule encoding a peptide comprising amino acids 17 to 40 of the amyloid β-peptide, wherein the lysine residue at position 28 is replaced by a cysteine residue.

In a further preferred embodiment, the present invention relates to an isolated nucleic acid molecule encoding a peptide consisting of amino acids 17 to 40 of the amyloid β-peptide, wherein the lysine residue at position 28 is replaced by a cysteine residue.

The invention furthermore relates to vectors containing the nucleic acid molecules of the invention. Preferably, they are plasmids, cosmids, viruses, bacteriophages and other vectors usually used in the field of genetic engineering. Vectors suitable for use in the present invention include, but are not limited to the T7-based expression vector for expression in bacteria, the PMSXND expression vector for expression in mammalian cells and baculovirus-derived vectors for expression in insect cells. Preferably, the nucleic acid molecule of the invention is operatively linked to the regulatory elements in the recombinant vector of the invention that guarantee the transcription and synthesis of an RNA in prokaryotic and/or eukaryotic cells that can be translated. The nucleotide sequence to be transcribed can be operably linked to a promotor like a T7, metallothionein I or polyhedrin promoter.

In a further embodiment, the present invention relates to recombinant host cells transiently or stably containing the nucleic acid molecules or vectors of the invention. A host cell is understood to be an organism that is capable to take up in vitro recombinant DNA and, if the case may be, to synthesize the peptides encoded by the nucleic acid molecules of the invention. Preferably, these cells are prokaryotic or eukaryotic cells, for example mammalian cells, bacterial cells, insect cells or yeast cells. The host cells of the invention are preferably characterized by the fact that the introduced nucleic acid molecule of the invention either is heterologous with regard to the transformed cell, i.e. that it does not naturally occur in these cells, or is localized at a place in the genome different from that of the corresponding naturally occurring sequence.

A further embodiment of the present invention relates to isolated peptides which are encoded by the nucleic acid molecules of the invention, as well as to methods for their production, whereby, e.g., a host cell of the invention is cultivated under conditions allowing the synthesis of the peptide and the peptide is subsequently isolated from the cultivated cells and/or the culture medium. Isolation and purification of the recombinantly produced peptides may be carried out by conventional means including preparative chromatography and affinity and immunological separations involving affinity chromatography with monoclonal or polyclonal antibodies. Accordingly, the present invention relates to peptide comprising amino acids 17 to 40 of the amyloid β-peptide, wherein the peptide is selected from the group consisting of
(a) a peptide wherein at least one of the naturally occuring amino acids is replaced by a cysteine residue;
(b) a peptide wherein at least one cysteine residue is inserted into the naturally occuring amino acid sequence; and
(c) a peptide which is a fragment, derivative or allelic variation of a peptide of (a) or (b).

The definitions given above for “fragment”, “derivative” or “allelic variation” also apply here.

As used herein, the term “isolated peptide” includes peptides substantially free of other peptides, proteins, nucleic acids, lipids, carbohydrates or other materials with which it is naturally associated. Such peptides, however, not only comprise recombinantly produced peptides but include synthetically produced peptides, i.e. peptides which have the same amino acid sequence, but which have been synthesized by standard methods, e.g., solid-phase synthesis.

The present invention also relates to an isolated homodimer of the above peptide containing at least one disulfide bond. Covalent coupling of two monomers via a disulfide bond can be carried out by the person skilled in the art according to well known methods, e.g., the methods described in Example 1(E), below.

The present invention also provides an isolated antibody which can specifically bind to the homodimer of the above peptide but, preferably, not to the monomeric form. The term “antibody”, preferably, relates to antibodies which consist essentially of pooled monoclonal antibodies with different epitopic specificities, as well as distinct monoclonal antibody preparations. Monoclonal antibodies are made from the peptide of the invention used as an antigen by methods well known to those skilled in the art (see, e.g., Köhler et al., Nature 256 (1975), 495). As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al., J. Nucl. Med. 24: 316 325 (1983).) Thus, these fragments are preferred, as well as the products of a FAβ or other immunoglobulin expression library. Moreover, antibodies of the present invention include chimerical, single chain, and humanized antibodies.

In a preferred embodiment, the antibody is the monoclonal antibody MabMX-O₂/MX-03 or the polyclonal sera MX-O₂/MX-03.

The present invention also relates to a method for diagnosing a disease associated with the presence of an enhanced amount of amyloid β-peptide, preferably Alzheimer's disease, which comprises contacting a target sample suspected to contain enhanced amounts of APP homodimers and/or Aβ-dimers with a reagent, preferably an antibody, which specifically binds to APP homodimers and/or Aβ-dimers. The target cellular component, i.e. APP homodimers and/or Aβ-dimers, e.g., in biological fluids or tissues, may be detected directly in situ, (e.g., according to the Examples, below) or it may be isolated from other cell components by common methods known to those skilled in the art before contacting with a probe. Detection methods include immunoassays, Western blot and other detection assays that are known to those skilled in the art.

The reagents, e.g. an antibody, can be detectably labeled, for example, with a radioisotope, a bioluminescent compound, a chemiluminescent compound, a fluorescent compound, a metal chelate, or an enzyme.

The presence of the homodimers or Aβ dimers discussed above in tissues can be studied with classical immunohistological methods (Jalkanen et al., J. Cell. Biol. 101 (1985), 976-985; Jalkanen et al., J. Cell. Biol. 105 (1987), 3087-3096; Sobol et al. Clin. Immunpathol. 24 (1982), 139-144; Sobol et al., Cancer 65 (1985), 2005-2010). Other antibody based methods useful for detecting the (homo)dimers include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin. In addition to assaying levels of (homo)dimers in a biological sample, the (homo)dimer can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma. A protein-specific antibody or antibody fragment which has been labeled with an appropriate detectable imaging moiety, such as a radioisotope (for example, ¹³¹I, ¹¹²In, ⁹⁹ mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹ mTc. The labeled antibody or antibody fragment will then preferentially accumulate at the location of cells which contain the specific APP homodimer. In vivo imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982)).

The reagent specific for AAP-/Aβ-dimers is also useful for prognosis and for monitoring the progression of the disease.

For evaluating whether the concentration of AAP homodimers or Aβ dimers is normal or increased, thus indicative for the probability to develop the disease or for the presence of the disease, the measured concentration is compared with the concentration in a normal tissue and in body fluids.

The present invention also relates to a method for identifying a compound capable of inhibiting the homodimerization of APP comprising:
(a) contacting cells transfected with a DNA sequence encoding APP with a compound to be screened; and
(b) determining whether the compound effects the formation of dimers.

Such compound (inhibitor) can be, for instance, specific peptides. Such inhibitors comprise molecules identified by the use of the recombinantly produced APP, e.g. APP₆₉₅. The recombinantly produced APP can be used to screen for and identify inhibitors, for example, by exploiting the capability of potential inhibitors to prevent (homo)dimerization under appropriate conditions. Such inhibitors for the APP homodimerization are synthetic peptides which represent the dimerization site of the APP. It has been shown that peptides which comprise the residues 91-111 and 448-465 of APP inhibit the dimerization of APP18-350 if used in crosslinking experiments in combination. Furthermore, inhibitors of the APP homodimerization are synthetic peptides which represent the dimerization site of APLP. It could be shown that peptides of APLP1 and APLP2 which are homologous to residues 91-111 and 448-465 of APP inhibit the dimerization of APP18-350. Further inhibitors of the APP homodimerization are APP specific ligands which sterically inhibit the dimerization of APP. These are ligands which bind to the N-terminal of APP and their agonists. These APP ligands are metall ions, e.g. zinc or copper, and glucosaminoglycans, e.g. heparin. Further inhibitors of APP homidimerization are molecules which have such an influence on the cholesterol content of the cellular membrane that dimerisation of APP is inhibited. The reduction or removal of cholesterol leads to an inhibition of APP dimerisation. These kind of inhibitor molecules are e.g. the statins.

It has been shown by the inventors that the loop structure which results from the cysteins at positions 98 and 105 in the sequence region 91-111 is important for the homodimerisation of APP. This has been confirmed by the experiment (c.f. FIG. 11) showing that the synthetic loop peptide 91-111 of APP is able to inhibit the homodimerisation of APP18-350 in the crosslinking test which is described in Scheuermann et al., J. of Biol. Chem., Vol. 276, No. 36, pp. 33923-33929 (2001). Since the distance between the cysteins and the cysteins itself are conserved within the APP and APLPs, the homologous loop peptides from APLP1 and APLP2 have been produced and analyzed in the crosslinking test. Both peptides have been able to inhibit the homodimerization of APP18-350. Control peptides having serin residues instead of cysteines at the respective positions could not inhibit the dimerisation. This means that the loop peptide itself in view of its binding to the N-terminal domain of APP is a so-called “Cu agonist” which causes a monomerisation by changing the conformation of the APP molecule and, thus, can inhibit the amyloid production. A similar mechanism is described for chromogranin (Thiele et al., J. Biol. Chem. 273(2), pp. 1223-1231 (1998)). The disulfide-bonded loop of chromogranins which is essential for sorting to secretory granules mediates homodimerisation.

The screening for these molecules (inhibitors) involves producing appropriate cells which express APP, e.g. APP₆₉₅, either as a secreted protein or on the cell membrane. Preferred cells include cells from mammals, preferably human neuroblastoma cells with SH-SY5Y being the most preferred cell line. Cells expressing APP (or cell membrane containing the expressed APP) are then preferably contacted with a test compound potentially containing the molecule to observe inhibition of homodimerization.

Alternatively, the assay can be carried out using cell-free preparations with APP affixed to a solid support, chemical libraries, or natural product mixtures. Such an in vitro test system can be established according to methods well known in the art. The assay may also simply comprise the steps of mixing a candidate compound with a solution containing APP, measuring inhibition of homodimerization and comparing the inhibition with a test system where the candidate compound is not present. Suitable in vitro test methods are gel permeation chromatography to detect the extent of dimerisation/monomerisation and BIACORE to detect the monomerisation of APP on the surface if inhibitors had been administered before.

Such screening for molecules could easily performed on a large scale, e.g. by screening candidate molecules from libraries of synthetic and/or natural molecules. Such an inhibitor is, e.g., an inorganic compound, a synthetic organic chemical, a natural fermentation product, a substance extracted from a microorganism, plant or animal, or a peptide. Additional examples of inhibitors are specific antibodies, preferably monoclonal antibodies.

Preferably, an ELISA assay can measure the level of homodimerization in a sample (e.g., biological sample) using a monoclonal or polyclonal antibody of the invention. The antibody can measure the level of homodimerization, directly or indirectly.

All of these above assays can be used to identify molecules useful as diagnostic or prognostic markers. The molecules discovered using these assays can also be used to treat disease or to bring about a particular result in a patient by inhibiting the formation of amyloid β-peptide.

The present invention also relates to a method of identifying a therapeutic agent comprising the steps of the above screening method; and
(i) synthesizing the compound obtained or identified in steps (a) and (b) of the above screening method or an analog or derivative thereof in an amount sufficient to provide said agent in a therapeutically effective amount to a patient; and/or
(ii) combining the compound obtained or identified in steps (a) and (b) or an analog or derivative thereof with a pharmaceutically acceptable carrier.

The present invention also relates to an inhibitor obtainable by the above method and to pharmaceutical compositions containing such an inhibitor.

The present invention also relates to a method for preventing, treating, or ameliorating a disease associated with the presence of an enhanced amount of amyloid β-peptide which comprises administering to a mammalian subject a therapeutically effective amount of a compound which decreases or inhibits homodimerization of APP. Preferred examples of such compounds are (a) antibodies specifically binding to APP momomers, (b) APLP1, (c) APLP2 and (d) the inhibitors described above. For administration, these compounds are preferably combined with suitable pharmaceutical carriers. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Administration of the suitable compositions may be effected by different ways, e.g. by intravenous, intraperitoneal, subcutaneous, intramuscular, topical or intradermal administration. The route of administration, of course, depends on the nature and stage of the disease and the kind of compound contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind and stage of the disease, general health and other drugs being administered concurrently.

For use in the diagnostic research discussed above, kits are also provided by the present invention. Such kits are useful for the detection of APP homodimers as a target cellular component, said kits comprising a probe for detection of APP homodimerization. The probe can be detectably labeled. In a preferred embodiment, said kit contains an antibody as described above and allows said diagnosis, e.g., by ELISA and contains the antibody bound to a solid support, for example, a polystyrene microtiter dish or nitrocellulose paper, using techniques known in the art. Alternatively, said kits are based on a RIA and contain said antibody marked with a radioactive isotope. In a preferred embodiment of the kit of the invention the antibody is labeled with enzymes, fluorescent compounds, luminescent compounds, ferromagnetic probes or radioactive compounds. The kit of the invention may comprise one or more containers filled with, for example, one or more probes of the invention. Associated with container(s) of the kit can be a notice in the form prescribed by a governmental agency regulating the manufacture, use or sale of pharmaceuticals or biological products, which notice reflects approval by the agency of manufacture, use or sale for human administration.

### EXAMPLES

The following Examples are intended to illustrate, but not to limit the invention. While such Examples are typical of those that might be used, other methods known to those skilled in the art may alternatively be utilized.

### Example 1

### General Methods

### (A) Expression Vectors, Site-Directed Mutagenesis for Receptor Dimerization Mutants

APP mutations were introduced by PCR amplification using the megaprimer method (Seraphin and Kandels-Lewis, Nucleic Acid Res. 24 (1996), 3276-7). Two outside primers spanned the BglII site(forward: TCG GCC TCG TCA CGT GTT C) and Xmnl site (reverse: CAA CTG GCT AAG GGG CTA TGT G) in APP while the reverse mutagenic primers introduced mutations at position 624 of APP₆₉₅ (K624A: CCA ATG ATT GCA CCT GCG TTT GAA CCC; K624R: CCA ATG ATT GCA CCC CGG TTT GAA CCC; K624C CCA ATG ATT GCA CCA CAA TTC GAA CCC). For cloning of the mutated DNA fragment the restriction enzymes XhoI and ClaI were used. The resulting cDNAs were cloned into the mammalian expression vector pCEP4 (Invitrogen/ITC Biotechnology, Heidelberg, Germany) at SmaI and SalI sites.

The SP-GFP-APP₆₉₅ clone was prepared from plasmids pEGFP-C1 (Clontech, Heidelberg) and pSP65-APP₆₉₅ (Tienari et al., PNAS USA 94 (1997), 4125-30). The GFP plasmid was restricted with PstI, blunt-ended with Klenow and religated to PEGFP-CO. The signal peptide of APP was amplified by PCR using pSP65-APP₆₉₅ as template and pairs of primers containing an engineered BamHI site (antisense primer: GGG GGC TAG CTC TAG ACA CTCGCA CAG CAG CGC ACT CG; sense primer: GGG GGA TCC ACC GGT ACC TCC AGC GCC CGA GCC). The DNA fragment encoding the signal sequence was amplified in pBluescript (Stratagene, Heidelberg) inserted into BamHI/SmaI sites) to pBS-SP. The GFP containing pBluescript plasmid pBS-SP-GFP-APP was cloned in 3-way ligations using three inserts and restriction sites XbaI/ClaI of pBS (insert 1: pBS-SP fragment XbaI/AgeI [derived from pBS-SP vector]; insert 2: pEGFP-CO fragment AgeI/LKpnI; insert 3: pSP65-APP₆₉₅ fragment KpnI/ClaI). The DNA sequence encoding the GFP-APP₆₉₅ fusion protein was ligated into pCEP4 cut with NotI/XhoI.

The mutant construct SP-GFP-APP₆₉₅/K624C was cloned into the pCEP4 vector using the sites NsiI and PmlI. All constructs were verified by sequencing.

(B) Cell Culture and Transfections

The pCEP4 vectors SP-GFP-APP₆₉₅, SP-GFP-APP₆₉₅-K624C and the N-terminal c-Myc-tagged APP₆₉₅ and APP₆₉₅-K624C (Tienari et al., PNAS USA 94 (1997), 4125-30; Peraus et al., J. Neurosci. 17 (1997), 7714-24) or the expression vector alone were transiently transfected into COS-7 cells in a lipofectamine-plus mixture (GIBCO BRL, Groningen, NL). Stably expressing cell lines have been obtained by transfecting the human neuroblastoma SH-SY5Y with the plasmids K624C, K624R and K625A containing the entire coding region of APP.

COS-7 cells were grown in Dulbecco-Vogt modified Eagle's medium supplemented with 10% calf serum (CS) under an atmosphere of 5% CO₂ at 37° C. Human neuroblastoma SY5Y cells were grown at 37° C. in minimal essential medium (MEM), supplemented with 10% calf serum, 2 mM L-glutamine, MEM non-essential amino acid mix and containing F-12. For stable expression, SY5Y were transfected with receptor cDNAs individually or in combination, selected with hygromycin (GIBCO) and maintained in media containing 0.3 μg/ml hygromycin). The properties of at least three different clonal lines were examined so that the influence of clonal variation on the observed phenotype could be determined.

(C) Metabolic Labeling

Transfected cells of a 100 mm dish grown to 70-90% confluence were washed with PBS and then treated with 3 ml MEM lacking methionine (Sigma, MUnchen) for 30-45 min. Labeling was performed in 3 ml of MEM lacking methionine, 5% dialyzed HI-FCS (1 kDa cut oft), supplemented with 300 μCi of [³⁵S]methionine (Amersham, Freiburg) and 5% CO² for 4 h. Alternatively, cells were incubated in medium lacking methionine and sulfate, supplemented with 5% dialyzed HI-FCS (1 kDa cut off) and 750 μCi carrier-free [³⁵S] sulfate for 4 h.

(D) Recombinant APP₃₉₋₃₅₀

The construct, consisting of residues 18-350 of APP from APP₇₇₀ was expressed in Pichia pastoris as described (Rossjohn et al., Nature Struc. Biol. 6 (1999), 327-31). Briefly, culture supernatant containing recombinant APP from secreted expression was adjusted to 150 mM NaCl and applied onto a column of Q-Sepharose (XK-50/20, Pharmacia, Freiburg) equilibrated with buffer A (50 mM Tris-HCl, pH 6.8; 150 mM NaCl), eluted with 70% buffer B (50 mM Tris-HCl, pH 6.8; 1 M NaCl) at a flow rate of 10 ml/min and rechromatographed onto a second column of Q-Sepharose (XK 10/5). APP containing fractions were pooled, adjusted to 250 mM NaCl and loaded onto a Superdex 200 pg (XK 16/60) column. Fractions were eluted at 0.5 ml/min in 1×PBS. The fractions eluting from an ion-exchange and a subsequent size-exclusion column were analyzed for APP content by Western blotting using monoclonal 22C11 as antibody. Protein concentrations were determined using the Bradford assay (Biorad, Mu\ddot{n}chen)

(E) Chemical Cross-linking

Chemical cross-linking reagents, bis (sulfosuccinimidyl) suberate (BS³), and dithiobis (sulfosuccinimidyl) propionate (DTSSP) were purchased from Pierce Chemical Co. (Rockford, Ill., USA), DTSSP is thiol cleavable, whereas BS³ is noncleavable.

Cells were grown to confluence in 100 mm dishes, washed in PBS, harvested, resuspended in lysis buffer (250 mM sucrose; 5 mM HEPES/NaOH, pH 7.4; 1 mM MgCl₂; 10 mM KCl) and homogenized. Crude membranes were obtained by centrifugation at 800 g and resuspended in cross-linking buffer (150 mM NaCl; 20 mM HEPES/NaOH, pH 7.5). Freshly prepared cross-linker solution was added and the suspension incubated for 1 h at RT with continuous end-over-end rotation. To quench residual cross-linker activity, the solution was adjusted to 50 mM Tris-HCl (pH 7.5) and the incubation continued for 30 min at RT. To analyze the tetrameric, dimeric and monomeric forms of APP, detergents Nonidet P-40 and Triton X-100 were added (to final concentrations of 2%) plus protease inhibitors (Complete tablets, Roche Molecular Biochemicals, Mannheim). The polyclonal antiserum 22734 was used to immunoprecipitate APP as described (Borchardt et al., Biochem. J. 344 (1999), 461-467). Crosslinking of recombinant APP₁₈₋₃₅₀ was performed by using the nonreversible, homobifunctional cross-linking agent BS³ or the cleavable cross-linker DTSSP (10 mM in water) in concentrations of 0.1-1 mM, depending on experimental requirements. To 10 μg (200 μg/ml in PBS) of purified APP₁₈₋₃₅₀ freshly prepared cross-linker was added and cross-linking allowed to proceed for 45 min at RT. The reaction was terminated by the addition of Tris-HCl (100 mM, pH 7.5). Treated and control samples were analyzed by SDS-PAGE and immunoblotting with polyclonal APP antiserum or monoclonal 22C11 (Hilbich et al., J. Biol. Chem. 268 (1993), 26571-7).

(F) Antisera and Electrophoresis

Monoclonal APP antibody 22C11, which recognizes the N-terminus of APP, and antibody WO-2, which recognizes the amino-terminal region of Aβ, have been described previously (Hilbich et al., J. Biol. Chem. 268 (1993), 26571-7; Ida et al., J. Biol. Chem. 271 (1996), 22908-14). Polyclonal APP antibodies 22734 were raised against recombinant APP from bacteria (22734) (Borchardt et al., Biochem. J. 344 (1999), 461-467), or raised against yeast APP₁₈₋₃₅₀ (polyclonal 40090) as described earlier for recombinant APP from bacteria (Weidemann et al., Cell 57 (1989), 115-26; Beher et al., J. Neurochem. 72 (1999), 1564-73). Immunoprecipitation with polyclonal rabbit APP antiserum and monoclonal Aβ antiserum, and immunodetection of APP were performed as described previously (Borchardt et al., Biochem. J. 344 (1999), 461-467; Jensen et al., Mol. Med. 6 2000), 291-302). For Western blotting of APP, samples were separated on 7.5% Tris-glycine or 7% Tris-tricine gels and immunodetected with mAb 22C11. Aβ was separated by 4-12% Bis-Tris gels (NUPA-GE, Novex, Groningen, NL) and bands were quantified with the Fuji-Bas PhosphorImager System. Data are averages for at least three separate trials.

After electrophoresis, dried gels were autoradiographed to help the excision of radiolabeled APP from the gel. Protein was eluted overnight from gel slices in 0.2M NH₄HCO₃ with 0.1% SDS at 37° C. To increase the yield, gel slices were transferred into 0.2M NH₄HCO₃/30% acetonitrile with 0.1% SDS and repeatedly extracted for 3 h at 37° C. The pooled samples were dried under vacuum and stored at −20° C. before electrophoresis.

### Example 2

### APP can Oligomerize In Vivo

In initial studies a conserved motif in the carbohydrate domain of recombinant APP for collagen binding and APP-APP interactions that were found to be modulated by N-terminal Zn(II), Cu(II) and heparin binding sites could be identified. To determine whether or not a monomer-dimer transition of APP also occurs in vivo, APP dimerization was analyzed and it could be shown for the first time that APP exists as dimer and higher oligomers held together by multiple non-covalent protein-protein interactions.

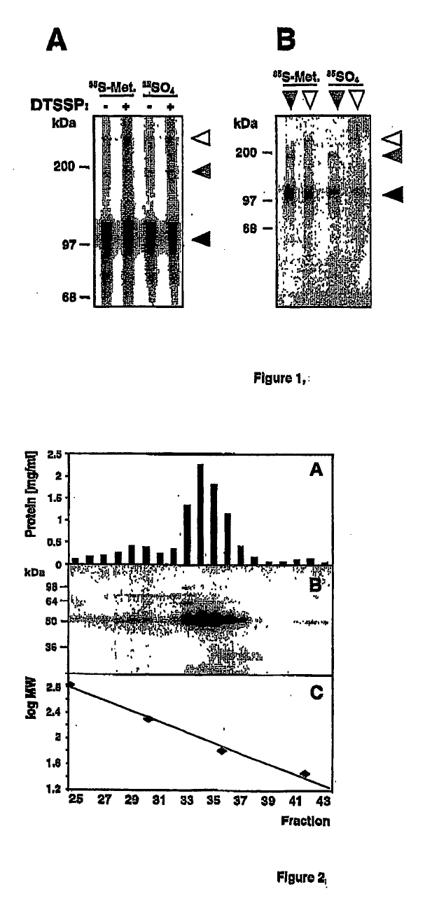

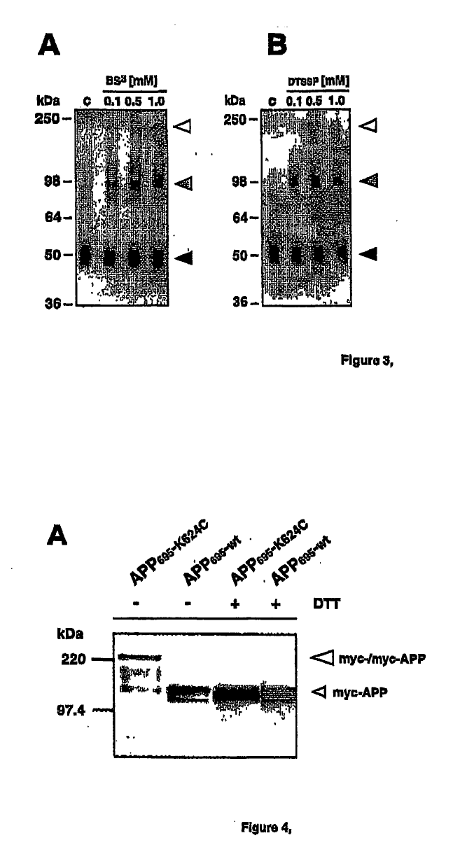

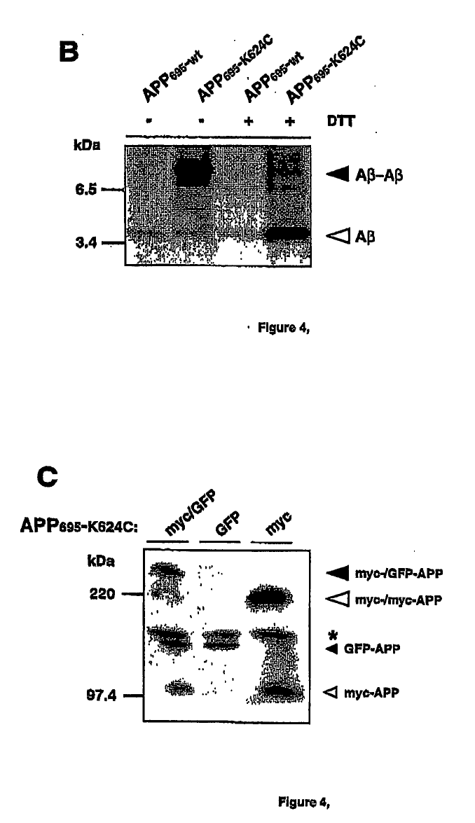

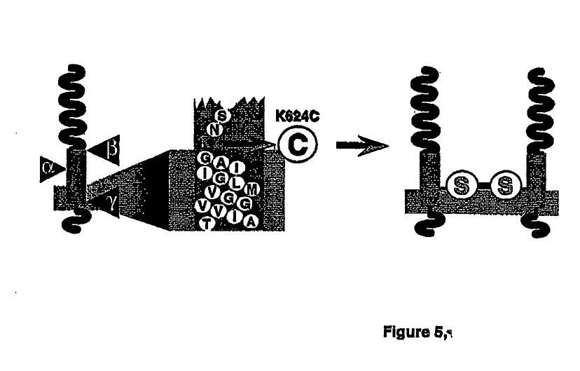

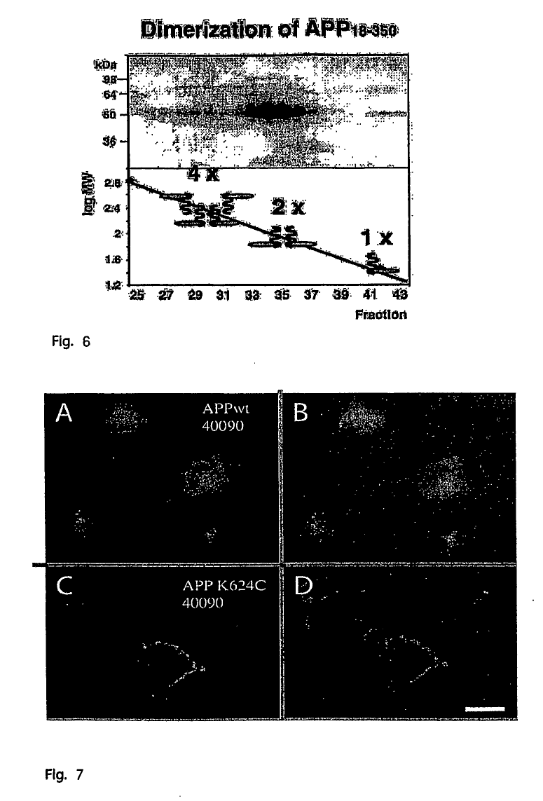

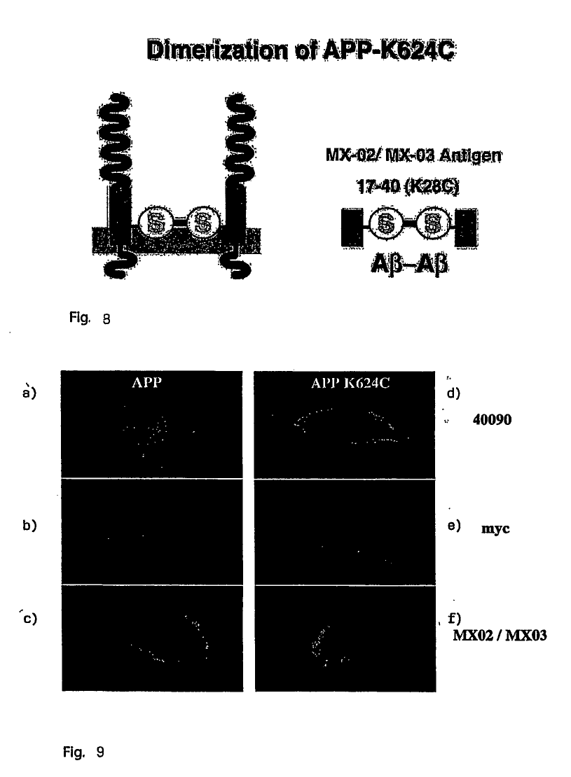

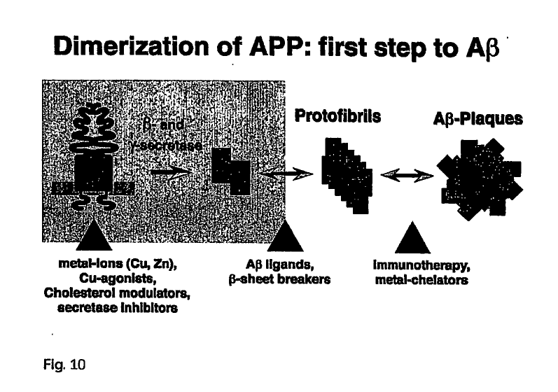

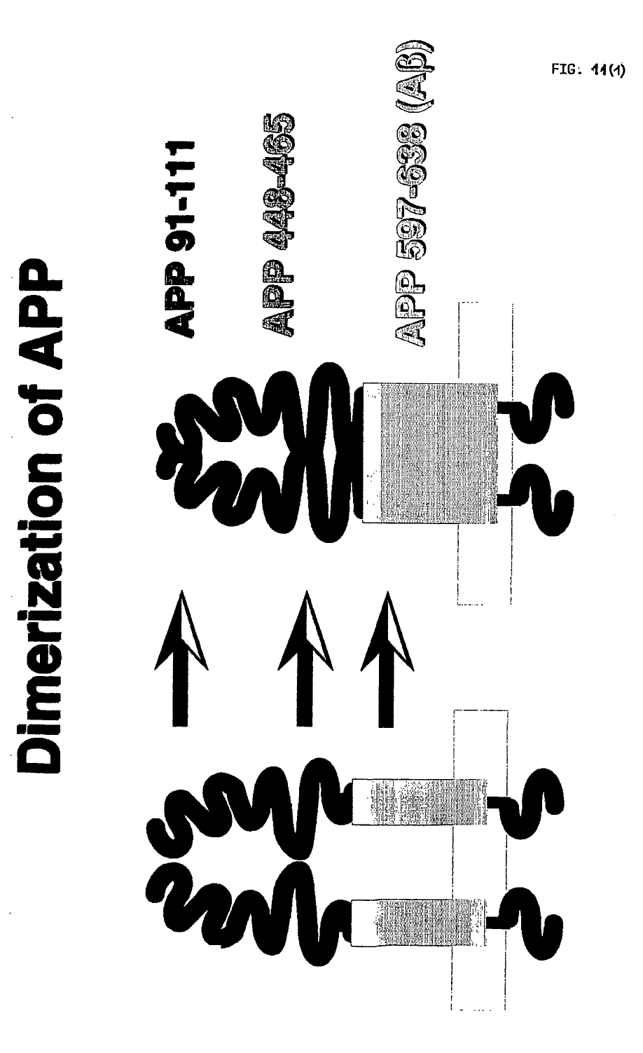

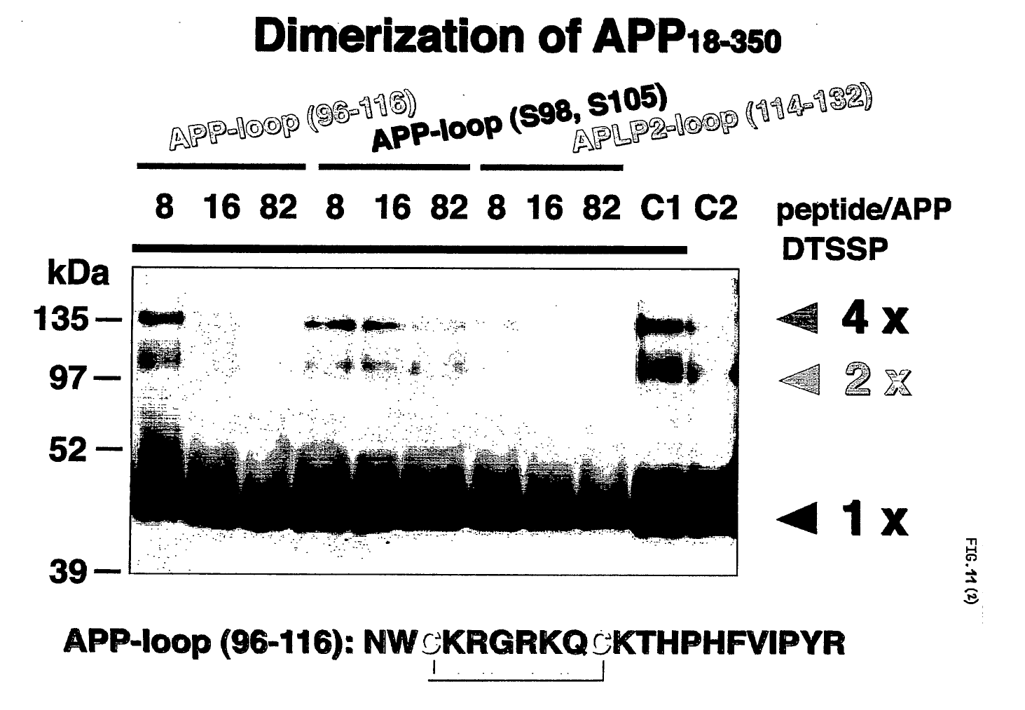

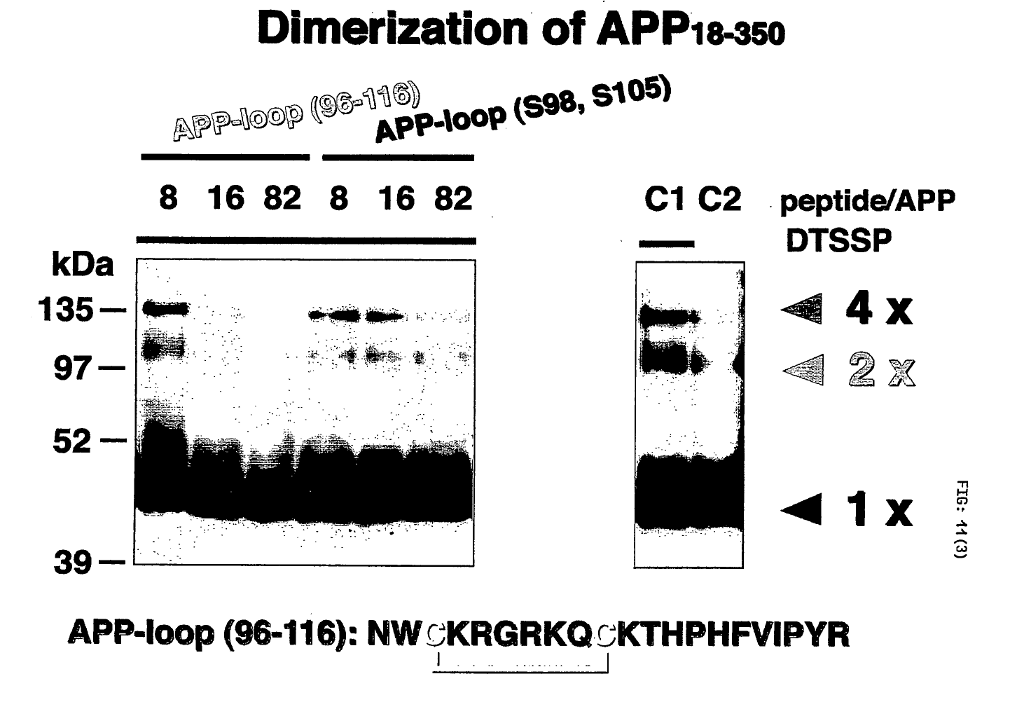

Normally, cellular APP migrates as a single band with a molecular mass of 110 kDa upon gel electrophoresis, irrespective of whether the protein was loaded with or without reducing agent. To confirm that APP can oligomerize in vivo, APP₆₉₅-transfected SH-SY5Y cells were labeled either with [³⁵S]methionine or [³⁵S]sulfate. Radiolabeled APP was immunoprecipitated from Nonidet P40 and Triton X-100 extracts and resolved by non-reducing/reducing SDS-PAGE (FIGS. 1A and B).

Addition of the cleavable cross-linker DTSSP to partially enriched APP resulted in strongly enhanced bands having the apparent molecular weight of APP₆₉₅ dimers and tetramers (FIG. 1A). Without addition of cross-linker, such bands were visible as pre-existing aggregates rather than functional dimers of APP obtained from crude membranes (FIG. 1A). This point of view is supported by the observation that cross-linked material did not all return to monomeric states after the cross-linker was reduced (FIG. 1B). Under non-reducing conditions, APP migrated at the monomeric molecular weight (110 kDa; FIG. 1A), whereas cross-linked APP migrated as dimers (about 19 okDa; FIG. 1A) and tetramers (about 40 kDa; FIG. 1A). To show that the APP dimers and tetramers were not covalently cross-linked and the cross-linking was not enzymatically induced by the tissue culture cells used, the different molecular forms of APP were isolated from the gel by preparative electroelution in a second experiment. APP complexes eluted from the gel were treated with DTT to cleave DTSSP bonds before running on a second SDS-PAGE, and FIG. 1B shows APP forms of each eluted fraction from the experiment 1 (FIG. 1A). As a control, size-matched gel pieces were eluted from untreated samples and failed to show any protein in electroeluted fractions from experiment 1 (data not shown).

In the presence of DTT and independent from the labeling technique, APP complexes were almost completely disrupted into monomers, with remaining original material (possibly resulting from preaggregated material) migrating either at the position of dimers or tetramers, respectively (FIG. 1B). Since we were unable to detect the presence of any proteins other than APP itself in the oligomers (even nonspecific cross-linking to other membrane proteins can be ruled out at 1 mM DTSSP), this suggests that oligomerie APP normally exists in SH-SY5Y cells as well-defined dimers and tetramers.

Most interestingly and as implied by the [³⁵S] sulfate labeling of APP complexes (FIGS. 1A and B), the oligomerization of APP occurs as early as in the ER, before [³⁵S]methionine labeled APP complexes are exported and before maturation of N-Iinked oligosaccharides and tyrosine sulfation of APP occurs. It should be emphasized that the APP oligomers formed in the ER represent immature forms. This is most evident from FIG. 1B, where monomerized [³⁵S]sulfate labeled APP migrates slower than [³⁵S]methionine labeled APP.

### Example 3

### APP has Oligomerization Domains in Addition to the Collagen Binding Site (CBP)

To investigate the possibility that APP has oligomerization domains in addition to the collagen binding site (CBP) of APP, the oligomerization potential of APP₁₈₋₃₅₀ expressed in Pichia pastoris by dynamic light scattering and size exclusion chromatography was tested. Using dynamic light scattering, mainly dimers were observed. The average diameter of aggregates was 4.6±0.1 nm at 20° C. For verification of existing APP₁₈₋₃₅₀ dimers, size exclusion chromatography was performed on a calibrated Superdex 200 pg XK16/60 FPLC column and fractions were analyzed for protein content (FIG. 2A). The advantage of this technique is that it reveals the molecular weight of native proteins. The column was equilibrated with protein standards as indicated in FIG. 2C. Recombinant yeast APP behaved anomalously upon gel filtration. As shown in FIG. 2A, the majority of APP eluted from gel filtration columns with a size of 113 kDa (fractions 33-36) which is much larger than expected for a monomer. A minor fraction of APP eluted at 267 kDa corresponding to tetramers (fractions 29 and 30). APP monomers were still observed with a molecular weight of 42 kDa (FIGS. 2A and C; fractions 41 and 42). This would be expected for a monomer-dimer equilibrium, which always has some unbound monomers. Because faster-moving dimers dissociating from the monomer pool are not observed (as it would be expected for a spontaneous monomer-dimer equilibrium), the equilibrium seems to be overbalanced in solution in favor of APP₈₋₃₅ₒ dimers (and significantly less strongly in favor of APP₁₈₋₃₅₀ tetramers).

Analysis of the eluted material by SDS-PAGE (FIG. 2B) and Western blotting showed that the major peak contained APP₁₈₋₃₅₀ with an apparent molecular mass of 5 okDa. SDS-PAGE under reducing and non-reducing conditions indicated the absence of any intermolecular disulfide-linked homo- or hetero-oligomers (data not shown). This is in agreement with biochemical and structural data suggesting that all cysteines of APP₁₈₋₃₅₀ are involved in disulfide bridging. Taken together with the results from the SDS PAGE (FIG. 2B), data from gel filtration experiments is consistent with the 113 kDa band being an APP₁₈₋₃₅₀ homodimer rather than a hetero-oligomer with another unknown protein.

To obtain additional evidence for the dimerization reaction of recombinant yeast APP18-350, the protein was cross-linked by adding the homobifunctional cross-linkers DTSSP or BS³, which both crosslink primary amines, i.e. predominantly lysine (Pierce, product description). No high molecular weight bands were observed without the cross-linkers (FIG. 3, lanes C). Additional bands at the molecular weight of APP₁₈₋₃₅₀ dimers and tetramers became visible when the samples had been incubated with the cross-linkers (FIG. 3). When 0.1 mM BS³ or 0.1 mM DTSSP was added, two additional bands appeared at 100 kDa and 200 kDa, respresenting dimeric APP₁₈₋₃₅₀ and tetrameric APP₁₈₋₃₅₀ (FIG. 3A). The distribution and intensity of those bands only slightly varied with the effective concentrations of BS³ and DTSSP implying that a stable monomer-dimer equilibrium was already reached at cross-linker concentrations of 0.1 mM (FIGS. 3A and B). Even in the presence of excess cross-linkers, the APP₁₈₋₃₅₀ monomer concentration remained constant and a change in the oligomeric state of APP₁₈₋₃₅₀ due to a shift in the ratio of dimeric to oligomeric forms could not be noticed (FIGS. 3A and B). This indicates that collisional crosslinks can be ruled out and only crosslinking of stable dimers was observed.

Thus, it can be concluded that at least two sites are involved in APP dimerization. The ectodomain of APP₆₉₅ showed an intrinsic ability to homodimerize through the collagen binding site in an earlier study and APP₁₈₋₃₅₀ (containing all 18 cysteines of full-length APP) has an even stronger dimerization potential than APP from SY5Y cell membranes (see band intensities in FIGS. 1 and 3) suggesting a zipper-like mechanism for dimerization of full-length APP.

### Example 4

### APP Dimerization Facilitates the Stabilization of Aβ Dimers that could Represent a Prelimiary Stage of beginning Fibrillogenesis

To corroborate the oligomeric state of cellular full-length APP and to analyze the influence of oligomerization on intracellular APP processing (i.e., amyloid Aα production), we wanted to generate covalently stabilized APP dimers with an intermolecular disulfide bond. A single unpaired Cys was introduced in the extracellular domain in the region immediately adjacent to the transmembrane domain of APP₆₉₅ (residues 625-648, encoded by exon 17). Because transmembrane domains are thought to emerge from the membrane in an helical conformation, the projected APP₆₉₅-K624C substitution (i.e., Aβ residue 28) was analyzed to fit with the predicted amphipathic interface in the APP juxtamembrane domain. The wild-type and the Cys-mutant APP695, which were overexpressed in SY5Y cells by stable transfection, all migrated at a relative molecular mass of about 110 kDa in SDS PAGE under reducing conditions (FIG. 4A). In contrast, all of the Cys-mutant proteins, but not the wild-type protein, migrated predominantly at about 220 kDa under non-reducing conditions (FIG. 4A, lane APP₆₉₅-K624C minus DTT). All mutant constructs investigated, K624C, K624R and K624A yielded the same amounts of secreted APP as in the wild-type transfected cells (data not shown) but dimers were only formed from the K624C construct. This indicates that the APP constructs were correctly expressed, fully glycosylated and transported and that the Cys-mutant efficiently dimerized in vivo by intermolecular disulfide formation (FIG. 5). Thus, APP₆₉₅ containing the K624C mutation in the extracellular domain dimerized constitutively via a disulfide bond, providing that the K624C mutant was most effective in stabilizing the APP dimer (FIG. 4A; FIG. 5). When corrected for the amount of secreted APP precipitated from the same sample, monomeric APP was produced to a similar extent from all APP forms (FIG. 4B, lanes APP₆₉₅-K624C and APP₆₉₅-Wt minus DTT). APP and Aβ levels from three independently generated APP₆₉₅ and APP₆₉₅-K624C cell lines were densitometrically calculated in the presence and in the absence of DTT and the ratio of Aβ/APP was determined. The ratio of Aβ/APP obtained from APP₆₉₅ cells was used as control and set at 100%. The evaluation resulted in an increase of monomeric Aβ under reducing conditions of 712.8±10%, 674.7±12% and 692.8±14% yielding a mean value of 693.4±12%. Thus, after conversion of dimeric Aβ to monomeric Aβ under reducing conditions (FIG. 4B, lanes APP₆₉₅-K624C and APP-wt plus DTT) the Aβ level was found to be increased by a factor of 7. By pulse-chase labelling experiments we found that neither the expression level of APP₆₉₅ and the K624C mutant varied, nor the half-life time of either of them was impaired (data not shown).

How could the 7-fold increased level of Aβ then interfere with APP processing? This could either be due to the APP₆₉₅-K624C mutant construct facilitating B-cleavage and attenuating α-cleavage, an overall increased expression of APP₆₉₅-K624C or inhibited Aβ clearance. To investigate if the ratio of β-secreted versus α-secreted soluble APP₆₉₅ derived from the K624C mutant was changed, myc-tagged APP₆₉₅-K624C was immunoprecipitated with rabbit polyclonal anti-c-Myc antibody (Borchardt et al., Biochem. J. 344 (1999), 461-467) and analyzed by monoclonal antibodies 22C11 (recognizing equally well sAPPα and sAPPβ due its epitope at the N-terminus of APP, and WO-2 (recognizing exclusively sAPPα). Since no differences between the total amounts of APP secreted (22C11 immunoreactivity of APP₆₉₅ versus APP₆₉₅-K624C mutant) and between sAPPα and sAPPB immunoreactivity could be detected, the possibility is favoured that dimerization of Aβ inhibits intracellular clearance. Consequently, it is attractive to postulate that APP dimerization facilitates the stabilization of Aβ dimers that could very well represent a preliminary stage of beginning fibrillogenesis.

The precipitation of soluble Aβ dimers from cell culture supernatant indicates that the disulfide bonds were maintained throughout the transport to the cell surface (FIG. 4B). The high yield and the release of Aβ dimers into the cell culture supernatant indicates that in the course of folding, disulfide bridges rearranged spontaneously by intramolecular thiol-disulfide exchange in the correct way.

It could be confirmed that the APP oligomers found were actually homodimers because myc-tagged APP₆₉₅-K624C could form homodimers (FIG. 4C; lane myc-K624C) that were never observed with GFP-tagged APP₆₉₅-K624C (FIG. 4C; lane GFP-K624C), possibly because of steric hindrance in the N-terminal dimerization domain of GFP-APP₆₉₅-K624C. The only heterodimer observed was formed between myc-tagged APP and GFP-tagged APP₆₉₅-K624C (FIG. 4C; lane myc-/GFP-K624C). Immunoblotting analysis showed that the myc-tagged APP₆₉₅-K624C was expressed at a higher level than the GFP-APP₆₉₅-K624C molecule but that the GFP-APP/Myc-APP heterodimer was preferentially formed in doubly transfected cells (FIG. 4C; lane myc-/GFPK624C). Thus, the present results also indicate that a dimer may only form when monomers in the proposed dimer are oriented in a particular geometry with respect to one another. The extended N-terminus of GFP-APP₆₉₅-K624C is inhibitory for dimerization with another GFP-APP₆₉₅-K624C molecule but not with another myc-APP₆₉₅-K624C (FIG. 4C) and although the predicted size of GFP-APP₆₉₅-K624C is higher it migrates faster than wild-type APP. Both observations can be explained by insights into the conformational changes of the protein backbone of APP that was gained when expressed APP fusion proteins were analyzed in E. coli. The characteristic aberrant migration of different naturally occuring isoforms of APP was found mainly to depend on aspartic and glutamic acid residues within the N-terminal domain of APP rather than on posttranslational modifications or the length of the protein (i.e., the number of amino acids).

To conclude, a dimerization-mediated regulation of APP metabolism is most likely to be physiologically relevant for the following reasons. Although the current model implies that APP dimerization does not serve as a signal to divert APP into the β-secretory pathway, APP may exist in the cell as populations of monomers and dimers in equilibrium. Ligand binding, for instance copper or zinc, can direct cellular APP into the α-secretory pathway. The inhibition of dimerization might be a possible mechanism. Proteins interacting with the intracellular C-terminal domain of APP, such as the neuronal adaptor protein X11α or presenilins, also could modulate the extent of dimerization of membrane bound APP. Upon internalization of APP monomers, such forms of APP would be less prone for Aβ production than internalized dimers.

Dimerization of APP matches a proposed signaling function of APP. APP is part of a G₀ protein-centered complex that transduces extracellular signals to the cytoplasm and the nucleus. If one assumes that binding of antibody 22C11 to the extracellular domain of APP (residues 66-81) exerts its activation of G₀ by a ligand mimetic mechanism, one must argue that monomeric APP may function as a G₀ protein-coupled receptor. This also indicates that as long as APP is not needed in its functional monomeric form, APP may exist as dimers in the absence of ligands. Thus, APP appears to be negatively regulated by dimerization as has been described for receptor like PTPs.

One of the questions that remain is whether other APLPs dimerize like APP. The conservation of the cysteine-rich N-terminal domain is consistent with the possibility that all APLPs can dimerize and the variable residues in this region could provide the necessary dimerization specificity. Additional data (not shown) indicate that APLP1 and APLP2 can form homodimers and that APP and APLP1 can form heterodimers. Accordingly, it is conceivable that heterodimerization of APP and APLP1 (or 2) can inhibit the formation of Aβ and that, thus, APLPs (and, in addition, further compounds blocking the APP homodimerization are possibly inhibitory compounds for Alzheimer's disease.

### Example 5

### Generation of Polyclonal sera MX-02/MX-03 Specifically Binding to APP/Aβ Homodimers

Polyclonal antibodies (MX02/MX03) against a synthetic peptide have been generated in two different rabbits. The peptide (p3-K28C) used for immunisation comprised the residues 17-40 (corresponding to the naturally occuring p3) of Aβ but had been modified at position 28 by replacing the lysine by a cysteine (analogous to the APP695-K624 construct, c.f. FIG. 8). Before the immunisation the peptide has been oxidized with air oxygen. The resulting dimer (Cys in position 28) has been purified by HPLC and only the dimerized peptide has been used for immunisation.
day 0: 1st. immunisation (1 mg antigen, Freund's adjuvans complete)
day 28: 2nd immunisation (0.5 mg antigen, Freund's adjuvans incomplete)
day 42: 3rd. immunisation (0.5 mg antigen, Freund's adjuvans incomplete)
day 56: 4th. immunisation (0.5 mg antigen, Freund's adjuvans incomplete)
day 70: 5th. immunisation (0.5 mg antigen, Freund's adjuvans incomplete)
day 84: dead of the animals

The sera of the rabbits have been stored by frozing.

The characterization of the obtained antisera has been done with the BIACORE technique. The test against the antigen p3-K28C has been made with monoclonal antibody G2-10 (ABETA GmbH, 69120 Heidelberg, Germany; specific for Aβ with 40 amino acid residues) recognizing the C-terminal residue of p3-K28C. Hence, the antibody G2-10 has been immobilized on a chip surface and loaded with p3-K28C. Then the sera have been injected. A strong immune reaction between the polyclonal antisera and the antigen p3-K28C could be observed.

A further characterization of the antisera MX-02/MX-03 has been done with APP transfacted COS-7 cells (c.f. FIG. 9). The immunostaining has been done according the general method described in Kins-Setal, Nat. Neurosci. 2000, 3, pp. 22-29. The immunostainings with MX02/MX03 gave an adequate result with APP695-K624C and wild type APP695 transfected COS7-cells. In both cases a clear staining of APP in the plasma membrane could be observed, although the staining was a little bit stronger when using APP695-K624C transfected cells. As control the polyclonal antibody 40090 (directed against APP dimers) has been used. This antibody showed plasma membran staining in APP695-K624C transfected cells but recognized only cellular APP (probably monomeric APP) in wild type transfected cells (c.f. FIGS. 6 and 7). In can be concluded that APP occurs naturally and dominantly as homodimer in the plasma membrane and that the polyclonal antibodies against Aβ dimers detect specific APP homodimers.

These results support that APP occurs in vivo as dimer in the plasma membrane and is the source for Aβ. The Aβ MX-02/MX-03 enable to detect APP forms specifically, i.e. APP dimers which are the precursors of Aβ.

The foregoing is meant to illustrate, but not to limit, the scope of the invention. The person skilled in the art can readily envision and produce further embodiments, based on the above teachings, without undue experimentation.

1-24. (canceled)

25. A method for identifying a compound capable of inhibiting the homodimerization of amyloid precursor protein (APP) and/or dimerisation of amyloid Aβ peptide (Aβ) comprising:
(a) contacting APP with a compound to be screened; and
(b) determining whether the compound effects the formation of APP homodimers.

26. The method of claim 25, wherein in step (a) cells transfected with a DNA sequence encoding APP are contacted with a compound to be screened.

27. The method of claim 25, wherein in step (a) the compound to be screened is mixed with a solution comprising APP.

28. The method of claim 25, wherein in step (a) APP is fixed to a solid support.

29. The method of claim 25, wherein APP comprises amino acids 17 to 40 of the amyloid β-peptide, wherein the lysine residue at position 28 is replaced by a cysteine residue.

30. An inhibitor of APP homodimerization obtainable by the method of claim 25.

31. The inhibitor of claim 30, wherein the inhibitor is a peptide comprising amino acid residues 91-111 or amino acid residues 448-465 of APP.

32. A method of identifying a therapeutic agent comprising the steps of the method of claim 25; and
(i) synthesizing the compound obtained or identified in steps (a) and (b) or an analog or derivative thereof in an amount sufficient to provide said agent in a therapeutically effective amount to a patient; and/or
(ii) combining the compound obtained or identified in steps (a) and (b) or an analog or derivative thereof with a pharmaceutically acceptable carrier.

33. An isolated nucleic acid molecule encoding a peptide comprising amino acids 17 to 40 of the amyloid β-peptide, wherein the lysine residue at position 28 is replaced by a cysteine residue.

34. A recombinant vector containing the nucleic acid molecule of claim 33.

35. An isolated peptide which is encoded by a nucleic acid molecule of claim 33.

36. A recombinant host cell that expresses the peptide of claim 35.

37. An isolated homodimer of the peptide of claim 35 containing at least one disulfide bond.

38. An isolated antibody specifically directed against the homodimer of claim 37.

39. The isolated antibody of claim 38 which is a polyclonal or monoclonal antibody.

40. A method for diagnosing a disease associated with the presence of an enhanced amount of amyloid β-peptide which comprises contacting a target sample suspected to contain enhanced amounts of APP homodimers and/or Aβ-dimers with an antibody which specifically binds to APP homodimers and/or Aβ-dimers.

41. The method of claim 40, wherein the disease is Alzheimer's disease.

42. The method of claim 40, wherein the antibody is detectably labeled.

43. The method of claim 42, wherein the label is selected from the group consisting of a radioisotope, a bioluminescent compound, a chemiluminescent compound, a fluorescent compound, a metal chelate, or an enzyme.

44. The method of claim 40, wherein the antibody is a polyclonal or monoclonal antibody.

45. A diagnostic kit comprising the antibody of claim 38.
